(19) United States
(12) Patent Application Publication
(10) Pub. No.: US 2025/0371457 A1
(43) Pub. Date: Dec. 4, 2025

(54) SYSTEMS AND METHODS FOR DYNAMICALLY OPTIMIZING ZONE CONFIGURATIONS

(71) Applicant: OPTUM, INC., Minnetonka, MN (US)

(72) Inventors: Jun Li, Minnetonka, MN (US); Daniel Peter Bloch, Burnsville, MN (US); Arun Kumar Tiwari, New Delhi (IN); Wataru Tamura, Murfreesboro, TN (US); Julie Zhu, Maple Grove, MN (US); Ayan Sengupta, Noida (IN); Kashish Minocha, Faridabad (IN)

(21) Appl. No.: 18/677,412

(22) Filed: May 29, 2024

(51) Int. Cl. G06Q 10/0631; G16H 40/20

(52) U.S. Cl. CPC G06Q 10/06312; G16H 40/20

(57) ABSTRACT

A computer-implemented method includes generating a plurality of zone configurations, at least by applying a machine learning (ML) model to demand node data indicating locations of a plurality of demand nodes; determining a plurality of respective costs for the plurality of zone configurations; selecting a particular zone configuration, from among the plurality of zone configurations, based on the plurality of respective costs; and storing in a memory one or more data objects representing the particular zone configuration.

### TECHNICAL FIELD

The present disclosure generally relates to dynamically grouping demand nodes into zones and assigning supply nodes to the zones in a manner that reduces supply node costs (e.g., travel time or distance).

### BACKGROUND

Home health care (e.g., in-home clinical exams and consultations) has undergone a massive expansion and is a huge cost for health providers and, ultimately, health care consumers. In-home medical exams are typically performed by an advanced practice clinician (APC). Because well-trained APCs are in very high demand, it is important to use them efficiently and maintain employee satisfaction. Sub-optimally deployed logistics solutions for these APCs can be the biggest driver of unnecessary travel and can negatively impact the APC's experience and job satisfaction. Properly designed field workforce optimization solutions can help minimize the total travel time or distance for the field workforce (APCs), which in turn helps maximize productive time and workforce satisfaction. Similar opportunities also exist in other (e.g., non-healthcare) scenarios where a supplier of a product or service is associated with multiple supply locations, and the product or service is to be delivered to customers or other recipients of the product or service who are distributed across multiple demand locations.

Conventional techniques for optimal assignment and routing of suppliers (e.g., APCs) to a plurality of destination nodes (e.g., patient locations) include the “Transportation Problem” and the “Traveling Salesman Problem.” The Transportation Problem generally addresses the transportation of something from m origins, O₁, . . . , Oₘ, to n destinations, D₁, . . . , Dₙ, with the objective of minimizing the total transportation cost. A related problem is the Traveling Salesman Problem, which can be formulated as: given a list of cities and the distances between each pair of cities, what is the shortest possible route for a supplier to visit each city exactly once and return to the origin city? However, conventional algorithms for solving the Transportation Problem and Traveling Salesman Problem are computationally expensive when the number of origins and destinations becomes large.

Zones (e.g., geographical areas) may be created to simplify the assignment of a plurality of suppliers) to a plurality of destinations. Conventional techniques for creating zones are sub-optimal, however, and tend to result in excessive travel time or distance for the suppliers in visiting their assigned destinations. Moreover, conventional zones are static, and not updated as destinations are added or removed. Furthermore, while conventional techniques may take supplier locations into account when allocating suppliers to pre-defined zones, such techniques fail to take supplier locations into account when initially defining the zones. As a result, suppliers may need to travel to zones that are further away from their starting points (e.g., homes or offices).

Accordingly, there is an opportunity for methodologies that optimize or otherwise improve zone configurations to reduce or minimize travel time or distance.

### BRIEF SUMMARY

The techniques disclosed herein generally relate to generating a configuration of zones (geographic areas), in which demand nodes are located, such that a system can assign supply nodes to the zones in a manner that reduces the total time or distance that suppliers must travel when servicing those demand nodes. In some embodiments, the system initially groups a plurality of demand nodes into a particular number of zones using partition clustering or other machine learning techniques, and then assigns one or more suppliers to each zone. In some embodiments, the system approximates supplier time or distance using a hub-and-spoke methodology, which assumes that the supplier travels from his or her supply node to a centroid of the zone and visits each demand node in the zone by traveling from the centroid to one demand node, and after visiting the demand node returns to the centroid before visiting the next demand node, etc. The system repeats the process for each of a number of different zone configurations. In some embodiments, for example, each zone configuration reflects a different number of zones (e.g., three, four, five, etc.). The system then selects a particular one of the zone configurations with its corresponding zones based on the total supplier times or distances for the zone configurations (e.g., by selecting the zone configuration that minimizes total supplier time and distance).

In one embodiment, a computer-implemented method includes: (1) generating, by one or more processors, a plurality of zone configurations, at least by applying a machine learning (ML) model to demand node data indicating locations of a plurality of demand nodes, wherein each zone configuration of the plurality of zone configurations includes a respective set of zones among a plurality of sets of zones; (2) determining, by the one or more processors, a plurality of respective costs for the plurality of zone configurations, wherein determining the plurality of respective costs for each zone configuration of the plurality of zone configurations comprises: (a) determining supply node allocations for the respective set of zones included in the zone configuration based on times or distances between (i) demand nodes, of the plurality of demand nodes, that are associated with the respective set of zones and (ii) supply nodes, of a plurality of supply nodes, that are eligible for the respective set of zones, and (b) determining a respective cost, of the plurality of respective costs, for the zone configuration based on (i) the times or distances and (ii) the supply node allocations; (3) selecting, by the one or more processors, a particular zone configuration, from among the plurality of zone configurations, based on the plurality of respective costs; and/or (4) storing in a memory, by the one or more processors, one or more data objects representing the particular zone configuration.

In one embodiment, a system includes: (A) a memory; (B) one or more processors communicatively coupled to the memory, the one or more processors configured to: (1) generate a plurality of zone configurations at least by applying a machine learning (ML) model to demand node data indicating locations of a plurality of demand nodes, wherein each zone configuration of the plurality of zone configurations includes a respective set of zones among a plurality of sets of zones; (2) determine a plurality of respective costs for the plurality of zone configurations, wherein determining the plurality of respective costs for each zone configuration of the plurality of zone configurations comprises: (a) determining supply node allocations for the respective set of zones included in the zone configuration based on times or distances between (i) demand nodes, of the plurality of demand nodes, that are associated with the respective set of zones and (ii) supply nodes, of a plurality of supply nodes, that are eligible for the respective set of zones, and (b) determining a respective cost, of the plurality of respective costs, for the zone configuration based on (i) the times or distances and (ii) the supply node allocations; (3) select a particular zone configuration, from among the plurality of zone configurations, based on the plurality of respective costs; and/or (4) store in the memory one or more data objects representing the particular zone configuration.

In one embodiment, one or more non-transitory computer-readable storage media include instructions that, when executed by one or more processors, cause the one or more processors to: (1) generate a plurality of zone configurations at least by applying a machine learning (ML) model to demand node data indicating locations of a plurality of demand nodes, wherein each zone configuration of the plurality of zone configurations includes a respective set of zones among a plurality of sets of zones; (2) determine a plurality of respective costs for the plurality of zone configurations, wherein determining the plurality of respective costs for each zone configuration of the plurality of zone configurations comprises: (a) determining supply node allocations for the respective set of zones included in the zone configuration based on times or distances between (i) demand nodes, of the plurality of demand nodes, that are associated with the respective set of zones and (ii) supply nodes, of a plurality of supply nodes, that are eligible for the respective set of zones, and (b) determining a respective cost, of the plurality of respective costs, for the zone configuration based on (i) the times or distances and (ii) the supply node allocations; (3) select a particular zone configuration, from among the plurality of zone configurations, based on the plurality of respective costs; and/or (4) store in a memory one or more data objects representing the particular zone configuration.

Advantageously, the disclosed methods, systems, and media provide improvements in computer functionality. Relative to processing techniques that attempt to reduce costs (e.g., reduce travel times or distances) without determining or using zones, the disclosed techniques can reduce costs in a manner that is faster and more computationally efficient. In particular, when using the disclosed methods, systems, and media to determine a zone configuration, the subsequent assignment of particular suppliers to particular demand nodes can make use of that zone configuration to constrain the complexity of the computational problem being solved.

Further, relative to techniques that do use zone configurations to reduce the complexity of the computational problem, the disclosed methods, systems, and media can generate zone configurations that enable a more efficient assignment/allocation of supplier nodes to demand nodes (e.g., assignments/allocations that result in lower total travel time or distance across all suppliers). In particular, the disclosed methods, systems, and media accomplish this at least by taking supply node locations into account at the zone configuration stage, rather than defining zones based solely on other factors such as demand node locations.

As is clear from the above and the description that follows, the present disclosure provides specific features that are not well-understood, routine, or conventional activity in the field (e.g., considering supply node locations when calculating costs for candidate zone configurations).

### BRIEF DESCRIPTION OF THE FIGURES

FIG. 1 depicts an example computing environment in which the techniques disclosed herein are implemented, according to one embodiment.

FIG. 2 depicts an example process for clustering demand nodes and assigning supply nodes, according to one embodiment.

FIGS. 3A-3D depict example clustering, zone partitioning, and supplier zone assignments, according to one embodiment and scenario.

FIG. 4 depicts a flow diagram of an example method for defining zones, according to one embodiment.

The figures depict example embodiments for purposes of illustration only. One skilled in the art will readily recognize from the following discussion that alternative embodiments of the systems and methods illustrated herein may be employed without departing from the principles of the disclosure described herein. While the disclosed techniques are primarily discussed herein with respect to a home health care use case, it is understood that the techniques may instead be applied to other use cases, such as shipment of goods.

### DETAILED DESCRIPTION

### Exemplary Computing Environment

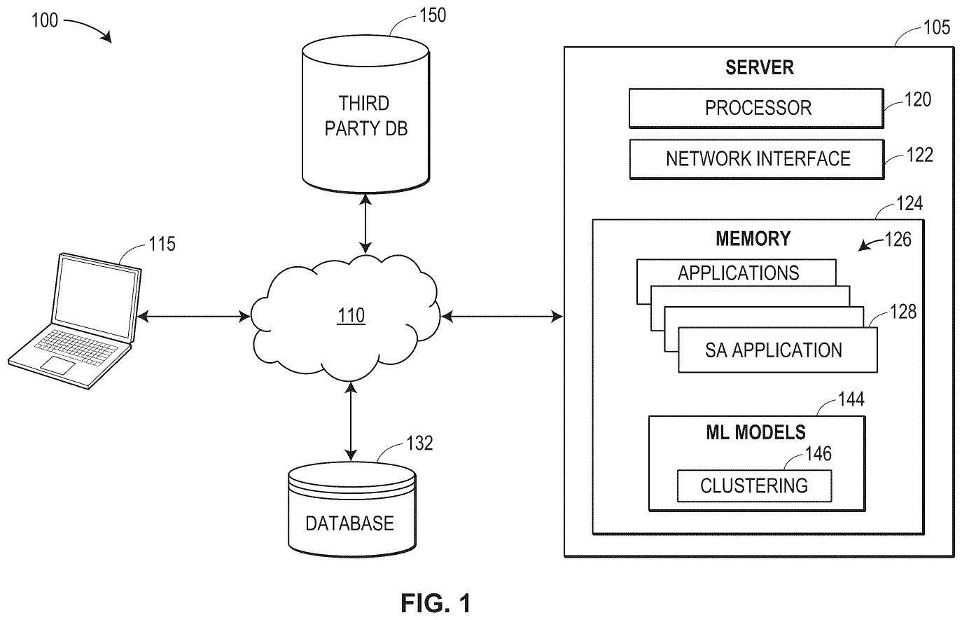

FIG. 1 depicts an embodiment of an example computing environment 100 in which techniques for generating recommended zones of demand nodes are implemented. Although FIG. 1 depicts certain entities, components, equipment, and devices, it should be appreciated that additional or alternate entities, components, equipment, and devices are also possible.

As illustrated in FIG. 1, the computing environment 100 includes, in one embodiment, a server 105 which can perform at least some of the functionalities and techniques disclosed herein. In one embodiment, for example, the server 105 is a server of a healthcare provider. In various embodiments, the server 105 includes only one server, or multiple servers that are co-located or remotely distributed. The server 105 may be part of a cloud network or may otherwise communicate with other hardware or software components within one or more cloud computing environments to send, retrieve, or otherwise analyze data or information described herein. In some example embodiments, the computing environment 100 comprises an on-premises computing environment, a multi-cloud computing environment, a public cloud computing environment, a private cloud computing environment, and/or a hybrid cloud computing environment.

The example computing environment 100 includes a network 110 comprising any suitable network or combination of networks, such as a local area network (LAN), a wide area network (WAN), the Internet, or a combination thereof. In one embodiment, for example, the network 110 includes a wireless cellular network (e.g., 4G, 5G, 6G, etc.). Generally, the network 110 enables bidirectional communication between the server 105 and/or a user device 115. In some embodiments, the server 105 is in communication with a user device 115. In one embodiment, the user device 115 is associated with an administrator generating zones and/or supplier assignments. In another embodiment, the user device 115 is associated with a supplier seeking his or her zone assignment.

In one embodiment, the network 110 comprises a cellular base station, such as cell tower(s), communicating with one or more other components of the computing environment 100 via wired/wireless communications based upon any one or more of various wireless communication standards, including NMT, GSM, CDMA, UMTS, LTE, 5G, 6G, or the like. Additionally or alternatively, the network 110 may comprise one or more routers, wireless switches, and/or other nodes communicating with the components of the computing environment 100 based upon any one or more of various other wireless and/or wired communications standards, including by non-limiting example, IEEE 802.11a/ac/ax/b/c/g/n (Wi-Fi), Bluetooth, Ethernet, and/or the like.

The example server 105 includes a processor 120. In some embodiments, the processor 120 includes one or more central processing units (CPUs) and/or graphics processing units (GPUs. In some embodiments, the processor 120 also includes one or more field programmable gate arrays (FPGAs), and/or any other suitable processor(s). The processor 120 is communicatively coupled to a memory 124 (e.g., via a computer bus not depicted in FIG. 1), such that the processor 120 can execute machine-readable instructions stored in memory 124 to perform any operations of the server 105 disclosed herein.

The server 105 includes a network interface 122, which enables the server 105 to communicate over the network 110 (e.g., with user device 115, and/or database 132 discussed below) via any suitable wired and/or wireless connection (e.g., according to any of the wired or wireless protocols/standards discussed above for network 110, and using any suitable controller(s) of the network interface 122).

In some embodiments, the memory 124 includes one or more memories and/or forms of volatile and/or non-volatile, fixed and/or removable memory, such as read-only memory (ROM), electronic programmable read-only memory (EPROM), random access memory (RAM), erasable electronic programmable read-only memory (EEPROM), and/or other suitable memory type(s) (e.g., hard drives, flash memory, MicroSD cards, etc.). The memory 124 stores machine-readable instructions executable by the processor 120, including the instructions of any of one or more application(s) 126. The memory 124 also stores an operating system (e.g., Microsoft Windows, Linux, UNIX, etc.) capable of facilitating the functionalities, applications, methods, or other software as discussed herein.

In the example embodiment of FIG. 1, the software applications 126 include a supplier assignment application (“SA application”) 128. The SA application 128 generally generates, assesses, and selects zone configurations for an environment that includes both supply nodes (e.g., health care providers or other suppliers) and demand nodes (e.g., patients or other customers or recipients). In some embodiments, the SA application 128 also allocates or assigns supply nodes to zones based on the selected zone configuration.

The example server 105 includes and/or has access to (e.g., via network 110 and a remote server) a database 132. In some embodiments, the database 132 includes a relational database, such as Oracle, DB2, MySQL, a NoSQL based database (e.g., MongoDB), or other suitable database(s). The database 132 generally stores data and/or datasets, such as demand node data (e.g., locations), supply node data (e.g., locations), training datasets used to train and/or operate one or more ML models, and so on. A dataset may include one or more types of data, records, files, etc. The terms “data” and “dataset” are used interchangeably herein. In alternative embodiments, the database 132 is stored locally in memory 124.

The memory 124 includes one or more ML models 144, which the SA application 128 employs to perform one or more clustering and/or other operations as described herein. In some embodiments, the ML models 144 include a clustering ML model 146. The clustering ML model 146 may be a k-means clustering model, a k-median clustering model, or a k-medoid clustering model, for example. Generally speaking, the clustering ML model 146 is trained (e.g., by the SA application 128, another application of server 105, or by another device or system) to receive data indicating locations of a plurality of demand nodes, and to cluster those demand nodes into a plurality of zones.

In some embodiments, the user device 115 comprises one or more computers and/or multiple, redundant, or replicated client computers accessed by one or more users. In some embodiments, the user device 115 includes one or more computing devices (e.g., desktop computer, laptop computer, terminal), mobile devices, wearables, smart watches, smart contact lenses, smart glasses, AR glasses/headsets, VR glasses/headsets, mixed or extended reality glasses/headsets, and/or other suitable electronic or electrical components. The user device 115 includes a memory and a processor for, respectively, storing and executing one or more modules, computer-executable instructions, etc. In some embodiments, the memory includes one or more suitable storage media such as a magnetic storage device, a solid-state drive, RAM, etc. The user device 115 includes a display, such as a graphical user interface (GUI) for presenting information to the user. The user device 115 accesses services or other functionalities and/or components of the computing environment 100 (e.g., of server 105) via the network 110. The user device 115 is generally used to request or receive information/data from, and or provide information/data to, one or more applications 126 of the server 105 (e.g., the SA application 128). In some embodiments, the computing environment 100 includes multiple user devices similar to the user device 115. In other embodiments, however, the computing environment 100 excludes any device similar to user device 115. For example, the server 105 may instead operate without providing any information to, and/or without receiving any information from, an external device or system.

In some embodiments, the server 105 also communicates with a third party database 150. The third party database 150 provides external data to the server 105. For example, the server 105 queries, using HTTP or an API, a third party server (not depicted) that hosts the third party database 150 and receives a response to the query. In one embodiment, the third party database 150 includes a geographical information system (GIS), such as ArcGIS. The server 105 provides addresses or latitude/longitude coordinates of locations to the third party database 150. The third party database 150 returns a time or distance between the locations.

### Exemplary Demand Node Clustering and Supply Node Assignment

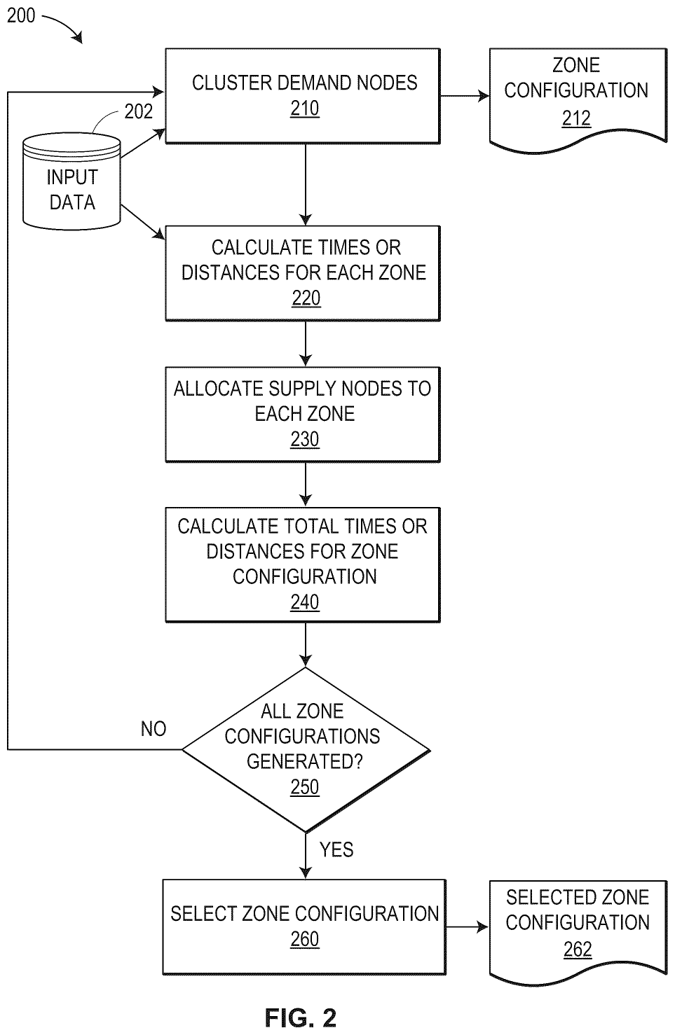

FIG. 2 depicts an example process 200 for clustering demand nodes into zones and assigning supply nodes. In some embodiments, the process 200 is performed (in part or entirely) by the SA application 128.

The example process 200 makes use of input data 202, which includes location data for supply nodes and demand nodes. The input data 202 is stored in the database 132 and/or third party database 150. For example, the input data 202 may include latitude/longitude coordinates of the supply nodes and demand nodes, or may include information from which such coordinates can be derived (e.g., residential addresses of supply nodes and demand nodes), etc. In another example, the input data 202 includes the latitude/longitude coordinates of the centers of the zip codes associated with the supply nodes and demand nodes. In some embodiments, the input data 202 includes data indicating one or more constraints, such as the geographic areas a supplier is allowed to serve (e.g., eligible zip codes, or maximum travel distances, etc.), and/or the maximum and/or minimum number of visits per time period (day, week, month, etc.) for a supplier, etc. The input data 202 can also include travel costs, e.g., travel times or distances, between locations (e.g., after such costs are calculated by the SA application 128 based on the respective supply and/or demand node locations).

Step 210 includes clustering demand nodes. In some embodiments, a user manually inputs a feasible range of zone counts k for a geographic area into the SA application 128. In some embodiments, clustering demand nodes includes the SA application 128 estimating feasible range of zone counts k, e.g., performing an “elbow method” on the within-cluster-sum-of-squares (WCSS) vs. zone counts k. For example, if the current solution has 4 zones, k may be 3-5 zones. Clustering demand nodes further includes generating a zone configuration 212 for a specific zone count k using unsupervised machine learning performed by the clustering model 146. In an embodiment, generating the zone configuration 212 includes, for a specific value of k, clustering the demand nodes by performing k-means clustering for that value of k, and calculating a zone centroid for each of the k clusters. In one embodiment, the zone centroid is the mean latitude/longitude of the demand nodes or zip codes allocated to the zone. In some embodiments, the zone configuration 212 includes a list of zip codes in each zone or zone boundaries defined by latitude/longitude coordinates.

Step 220 includes calculating a set of times or distances for each zone in the zone configuration 212. In some embodiments, the set of times or distances includes the time or distance between supply nodes eligible for the zone and the zone centroid. In some embodiments, the set of times or distances includes the time or distance between the demand nodes of the zone and the zone centroid. For example, step 220 may use a hub and spoke model, which assumes that the supplier will travel from the supply node to the zone centroid for the assigned zone, then travel to the first demand node of the zone and return to the zone centroid before traveling to the next demand node. This model assumes that the supplier will continue traveling to demand nodes and returning to the zone centroid until all visits are complete, and then will return back to the supply node. In some embodiments, the times or distances between nodes and the centroid are retrieved from the third party database 150.

Step 230 includes allocating one or more supply nodes from the eligible supply nodes to each zone of the zone configuration 212, based on the set of times or distances calculated at step 220. Allocating the one or more supply nodes includes minimizing the total travel times or travel distances of all the suppliers. In some embodiments, the SA application 128 allocates the supply nodes to the zones using integer programming techniques, where the decision variables are integers only. Allocating the one or more supply nodes at step 230 is subject to one or more constraints, in some embodiments, such as eligible geographic areas, the supplier's capacity, and/or minimum and maximum visits per supplier.

Step 240 includes calculating total travel times or distances for the zone configuration 212. In an embodiment, this includes, based on the set of times or distances calculated in step 220, summing the travel times or travel distances of all the allocated supply nodes for the zone configuration 212. In some embodiments, the total travel times or distances includes a commute cost plus a cost between visits. The commute cost is the sum of the travel time or distance between the allocated supply node to the zone centroid and back to the supply node for each allocated supply node, as given by the following equation:

$$CommuteCost = {\sum }_{SupplyNodes} Distance ( SupplyCode , Centriod ) * 2$$

The cost between visits is the sum of the times or distances to visit each demand node, assuming that the supplier returns to the centroid after visiting each demand node, i.e., the times or distances between the zone centroid and each of the demand nodes, as given by the following equation:

$$CostBtwVisits = {\sum }_{Zones} ( {\sum }_{DemandNodes} Distance ( DemandNode , Centroid ) * 2 )$$

Step 250 includes determining whether all zone configurations have been generated. The determining step includes evaluating whether one or more zone configurations have been generated for each value of k. If the answer is no, then k is incremented by one, and the process returns to step 210. If the answer is yes, then the process proceeds to step 260.

Step 260 includes selecting a selected zone configuration 262 (e.g., a best or optimal zone configuration). Selecting the selected zone configuration 262 includes identifying the zone configuration 212 that has the lowest total travel time or travel distance determined in step 240, while still satisfying business rules and requirements. The business rules and requirements exclude zone configurations 212 in which the number of demand nodes is below a minimum, the number of demand nodes is imbalanced between zones, or any other suitable rules or requirements. In some embodiments, step 260 includes updating a scheduling system with one or more data objects representing the selected zone configuration 262.

The zone configuration 262 may include data indicating the boundaries of the zones, the zip codes within the zones, the demand nodes assigned to each zone, and/or the supply nodes assigned to each zone, for example.

In alternative embodiments, the process 200 includes more, fewer, and/or different steps than those shown in FIG. 2.

Exemplary Clustering of Demand Nodes into Zones

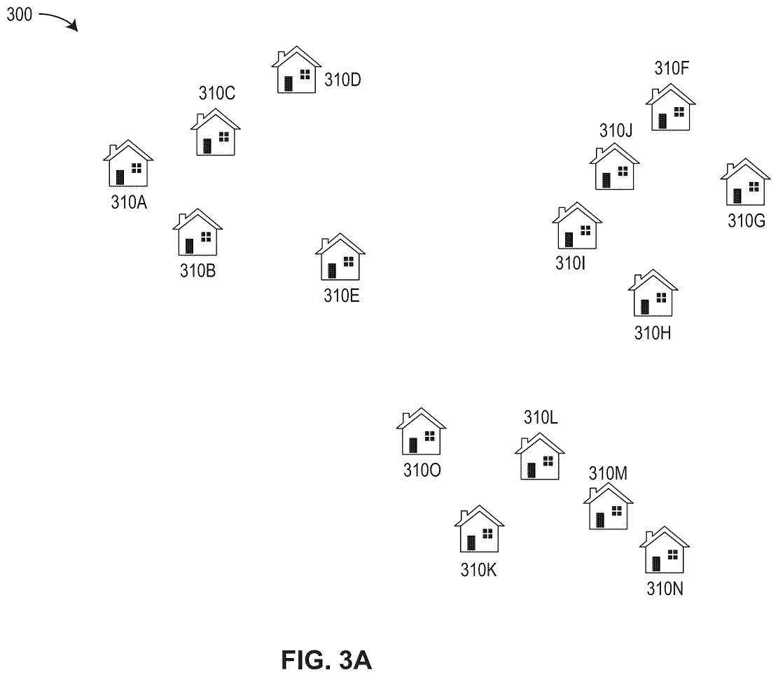

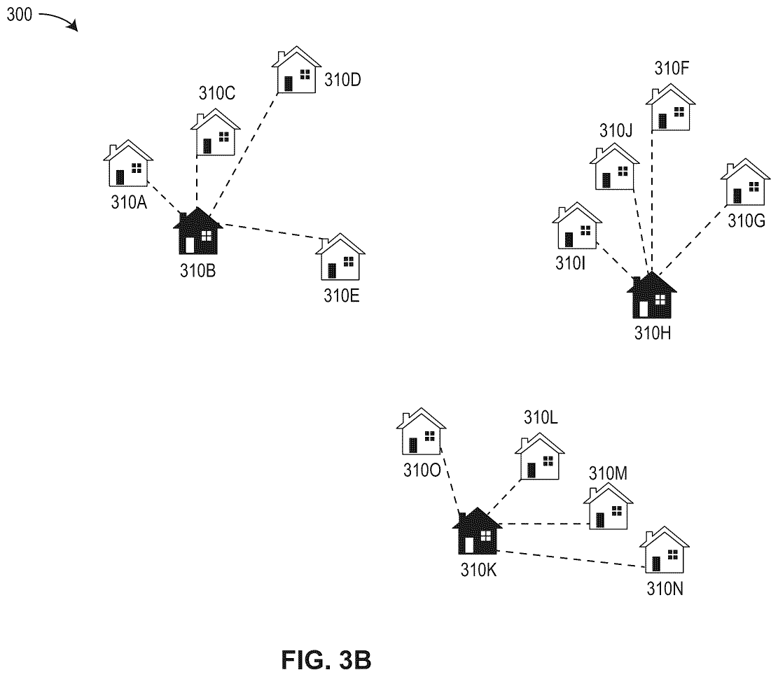

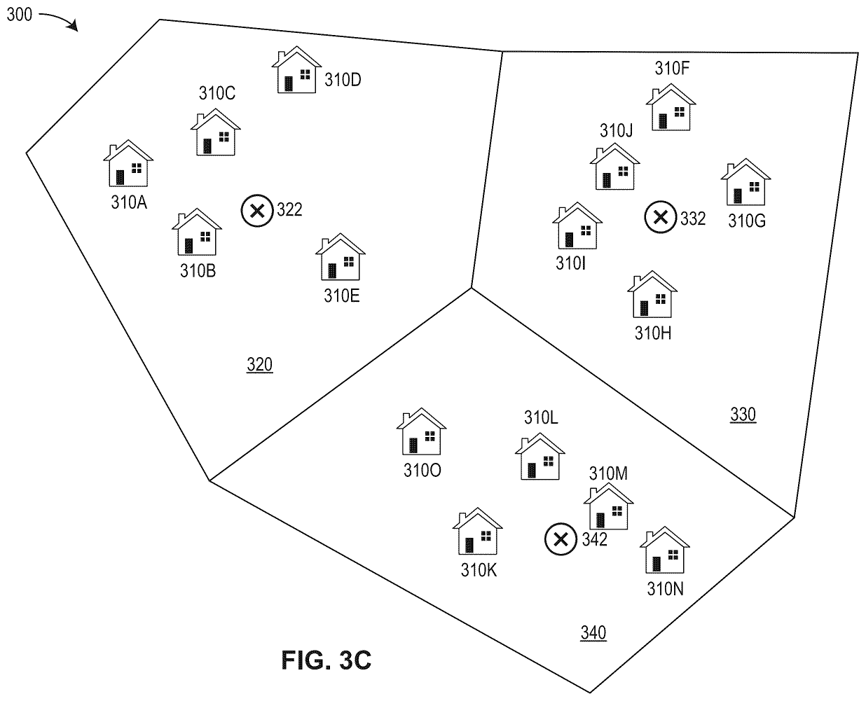

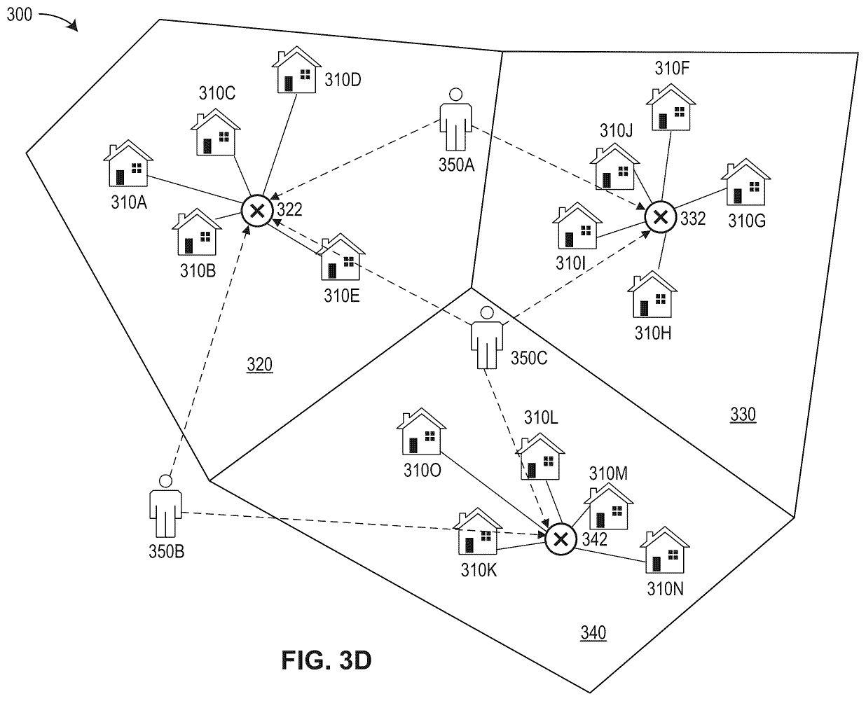

FIG. 3A illustrates an exemplary area 300 for which recommended zones of demand nodes are generated and suppliers are assigned the recommended zones. For example, the area 300 is associated with a city, county, state, territory, district, or any other suitable region. The area 300 includes a plurality of demand nodes 310A-3100. Each demand node 310A-3100 is associated with a location identifier, such as an address, zip code, or longitude/latitude coordinate.

FIG. 3B illustrates clustering of the demand nodes 310A-3100. In some embodiments, the SA application 128 provides a zone count k and location information for the demand nodes 310A-3100 as input to an ML model, such as clustering model 146. The ML model assigns k number of demand nodes 310A-3100 as centroids. In the illustrated example, the ML model has assigned three demand nodes, 310B, 310H, and 310K, as centroids. The ML model assigns the remaining non-centroid demand nodes to one of the k clusters based upon the shortest distance to the zone centroids. In some embodiments, the SA application 128 retrieves the distances between the demand nodes and the centroids from the third party database 150 and provides the distances as input to the ML model. In the illustrated example, the ML model has assigned demand nodes 310A and 310C-310E to centroid 310B, demand nodes 310F, 310G, 310I, and 310J to centroid 310H, and assigned demand nodes 310L-3100 to centroid 310K. For example, each cluster may have an equal or unequal number of demand nodes assigned. In some embodiments, the ML model is constrained by a cluster minimum location parameter Cₘᵢₙ such that the ML model is limited to outputting clusters having at least the Cₘᵢₙ number of demand nodes. In some embodiments, the ML model is constrained by a cluster maximum location parameter Cₘₐₓ such that the ML model is limited to outputting clusters having fewer than or equal to the Cₘₐₓ number of demand nodes.

FIG. 3C illustrates the calculation of cluster centroids and the division of the area 300 into zones 320, 330, and 340. In some embodiments, the ML model calculates the centroid or medoid of each cluster. The centroid or medoid represents the center of each cluster. The centroid or medoid may or may not be the same as the centroid assigned above. As illustrated, cluster 310A-310E has the centroid 322, cluster 310F-310J has the centroid 332, and cluster 310K-3100 has the centroid 342. In some embodiments, the ML model partitions the area 300 into zones, wherein each zone encompasses a cluster. As illustrated, zone 320 includes the cluster 310A-310E, zone 330 includes the cluster 310F-310J, and zone 340 includes the cluster 310K-3100. In some embodiments, the ML model determines the boundary between zones by bisecting the distance between two centroids, bisecting the distance between the two nearest demand nodes, or any other suitable method.

FIG. 3D illustrates determination of supplier assignments in the area 300. In the illustrated example, three suppliers 350A-350C are available for assignment. There may be more suppliers than zones such that there are two or more suppliers per zone. The SA application 128 determines supplier assignments by attempting to minimize distance or time for suppliers. In one embodiment, the SA application 128, using a “hub-and-spoke” model, adds the distances or times between the centroid and each demand node in a zone to calculate the cost between visits. The SA application 128 adds the distance or time of a supplier to the zone centroid to determine a commute cost. In some embodiments, the SA application 128 repeats the supplier cost calculations for a number of different supplier assignments or zone configurations with the objective of minimizing the total supplier cost. In some embodiments, the SA application 128 is constrained by a maximum supplier cost Sₘₐₓ, such that the SA application cannot output a supplier assignment in which a supplier's total distance or time exceeds Sₘₐₓ.

### Exemplary Method of Generating Zones and Supplier Assignments

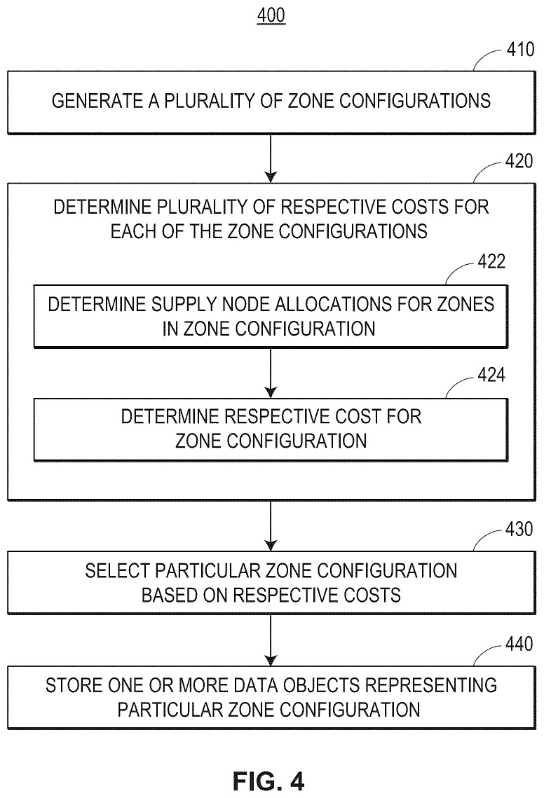

FIG. 4 depicts a flow diagram of an exemplary computer-implemented method 400 for generating zones and assigning supplier nodes. One or more (e.g., all) steps of the computer-implemented method 400 are implemented as a set of instructions stored on a computer-readable memory and executable on one or more processors. In some embodiments, the computer-implemented method 400 of FIG. 4 is implemented via a system, such as the server 105, user device 115, database 132, and/or the third party database 150. The computer-implemented method 400 operates in conjunction with one or more of the scenarios and/or environments illustrated in FIGS. 1-3D and/or in other environments. In some embodiments, for example, the computer-implemented method 400 is repeated as necessary, such as when supply nodes or demand nodes are added or removed.

The example computer-implemented method 400 includes at block 410 generating a plurality of zone configurations, at least by applying an ML model (e.g., the clustering model 146) to demand node data. For example, the ML model can be a clustering model, such as k-means, k-median, or k-medoid. In one embodiment, the demand node data is obtained from the database 132, third party database 150, or another suitable data source. In some embodiments, demand node data indicates the location of a plurality of demand nodes and includes one or more of addresses, zip codes, or latitude/longitude coordinates for the plurality of demand nodes. In some embodiments, the demand node data includes time or distances between the plurality of demand nodes.

Each zone configuration of the plurality of zone configurations includes a respective set of zones among a plurality of sets of zones. In one embodiment, each zone configuration consists of a different number of zones. For example, block 410 may include applying the clustering model to the demand node data N times, where Nis the number of zone configurations, and where each of the N zone configurations consists of a different number of zones.

The computer-implemented method 400 also includes, at block 420, determining a plurality of respective costs for the plurality of zone configurations. In the depicted embodiment, block 420 includes performing blocks 422 and 424 for each zone configuration of the plurality of zone configurations.

The computer-implemented method 400 includes at block 422 determining supply node allocations for the respective set of zones included in the zone configuration. In some embodiments, the supply node allocations are determined based on times or distances between (i) demand nodes, of the plurality of demand nodes, that are associated with the respective set of zones and (ii) supply nodes, of a plurality of supply nodes, that are eligible for the respective set of zones. In some embodiments, the allocation at block 422 attempts to minimize the times or distances using an objective function. In one embodiment, determining the supply node allocations includes verifying that the allocation does not exceed a maximum count for each supply node, where the maximum count is a maximum number of demand nodes that can be assigned to each supply node. In one embodiment, determining the supply node allocations includes verifying that the allocation equals or exceeds a minimum count for each supply node, where the minimum count is a minimum number of demand nodes that can be assigned to each supply node.

The computer-implemented method 400 includes at block 424 determining a respective cost, of the plurality of respective costs for the zone configuration. In some embodiments, determining the respective cost is based on (i) the times or distances and (ii) the supply node allocations.

The computer-implemented method 400 includes at block 430 selecting a particular zone configuration from the plurality of zone configurations based on the plurality of respective costs (e.g., selecting the zone configuration having the lowest cost).

In one embodiment, the computer-implemented method 400 includes at block 440 storing in memory one or more data objects representing the particular zone configuration. For example, the data objects include a list of addresses of demand nodes assigned to the supply nodes for the particular zone configuration.

In one embodiment, the computer-implemented method 400 also includes a block (not shown in FIG. 4) in which a zone assignment is determined for one or more of the supply nodes based on the particular zone configuration. For example, the zone assignment may include the zone and demand nodes assigned to the one or more supply nodes. In one embodiment, the computer-implemented method 400 includes causing a display to present a visual indication of the zone assignment. The visual indication includes a list of addresses of the assigned demand nodes, a map depicting the zone and demand nodes, or any other suitable representation. The visual indication is presented on a display of the user device 115, for example.

It should be understood that not all blocks of the computer-implemented method 400 are required to be performed. Moreover, the computer-implemented method 400 is not mutually exclusive (i.e., block(s) from computer-implemented method 400 may be performed in any particular implementation). Further, in some embodiments, the operations of the computer-implemented method 400 are not performed strictly in the order shown in FIG. 4. For example, portions of blocks 410, 420, 422, and/or 424 may all be performed in an iterative manner (e.g., determining supply node allocations and a respective cost for a first zone configuration before doing the same for a second zone configuration, and then perhaps for a third zone configuration, etc.).

### EXAMPLES

The following list of examples reflects a variety of the embodiments explicitly contemplated by the present disclosure. Those of ordinary skill in the art will readily appreciate that the examples below are neither limiting of the embodiments disclosed herein, nor exhaustive of all of the embodiments conceivable from the disclosure above, but are instead meant to be exemplary in nature.

Example 1. A computer-implemented method comprising: generating, by one or more processors, a plurality of zone configurations, at least by applying a machine learning (ML) model to demand node data indicating locations of a plurality of demand nodes, wherein each zone configuration of the plurality of zone configurations includes a respective set of zones among a plurality of sets of zones; determining, by the one or more processors, a plurality of respective costs for the plurality of zone configurations, wherein determining the plurality of respective costs for each zone configuration of the plurality of zone configurations comprises: determining supply node allocations for the respective set of zones included in the zone configuration based on times or distances between (i) demand nodes, of the plurality of demand nodes, that are associated with the respective set of zones and (ii) supply nodes, of a plurality of supply nodes, that are eligible for the respective set of zones, and determining a respective cost, of the plurality of respective costs, for the zone configuration based on (i) the times or distances and (ii) the supply node allocations; selecting, by the one or more processors, a particular zone configuration, from among the plurality of zone configurations, based on the plurality of respective costs; and storing in a memory, by the one or more processors, one or more data objects representing the particular zone configuration.

Example 2. The computer-implemented method of Example 1, wherein determining the supply node allocations for the respective set of zones included in the zone configuration based on the times or distances comprises: summing the times or distances from (i) the demand nodes to a zone centroid or a zone medoid for the respective set of zones and (ii) the supply nodes to the zone centroid or the zone medoid for the respective set of zones.

Example 3. The computer-implemented method of Examples 1 or 2, wherein the ML model comprises a k-means clustering model.

Example 4. The computer-implemented method of Examples 1 or 2, wherein the ML model comprises a k-median clustering model.

Example 5. The computer-implemented method of Examples 1 or 2, wherein the ML model comprises a k-medoid clustering model.

Example 6. The computer-implemented method of any of Examples 1 through 5, wherein each zone configuration of the plurality of zone configurations consists of a different number of zones.

Example 7. The computer-implemented method of Example 6, wherein the different number of zones is a manually-specified range of zone counts.

Example 8. The computer-implemented method of Example 6, further comprising: determining, by the one or more processors, a range of zone counts comprising the different number of zones using an elbow method.

Example 9. The computer-implemented method of any of Examples 1 through 8, wherein determining the supply node allocations for the respective set of zones included in the zone configuration comprises: verifying that an allocation of supply nodes to the respective set of zones does not exceed a maximum count for each supply node, wherein the maximum count is a maximum number of demand nodes that can be assigned to a single supply node.

Example 10. The computer-implemented method of any of Examples 1 through 9, wherein determining the supply node allocations for the respective set of zones included in the zone configuration comprises: verifying that an allocation of supply nodes to the respective set of zones equals or exceeds a minimum count for each supply node, wherein the minimum count is a minimum number of demand nodes that can be assigned to a single supply node.

Example 11. The computer-implemented method of any of Examples 1 through 10, wherein determining the supply node allocations for the respective set of zones included in the zone configuration comprises: verifying that an allocation of supply nodes to the respective set of zones does not exceed a maximum travel distance for one or more of the supply nodes.

Example 12. The computer-implemented method of any of Examples 1 through 11, wherein determining the supply node allocations for the respective set of zones included in the zone configuration comprises: verifying that an allocation of supply nodes to the respective set of zones complies with eligible geographic areas for each supply node.

Example 13. The computer-implemented method of any of Examples 1 through 12, further comprising: determining, by the one or more processors and for one or more of the supply nodes, a zone assignment based on the particular zone configuration, wherein the zone assignment comprises a single zone of the plurality of respective zones assigned to each of the one or more supply nodes; and causing, by the one or more processors, a display to present a visual indication of the zone assignment.

Example 14. The computer-implemented method of any of Examples 1 through 13, wherein determining the supply node allocations for the respective set of zones included in the zone configuration comprises minimizing a total cost for the zone configuration using an objective function.

Example 15. The computer-implemented method of any of Examples 1 through 14, wherein the demand node data comprises latitude/longitude coordinates of the demand nodes.

Example 16. The computer-implemented method of any of Examples 1 through 14, wherein the demand node data comprises addresses of the demand nodes.

Example 17. The computer-implemented method of any of Examples 1 through 14, wherein the demand node data comprises latitude/longitude coordinates of zip codes associated with the demand nodes.

Example 18. The computer-implemented method of any of Examples 1 through 17 further comprising: updating, by the one or more processors, a scheduling system with the one or more data objects.

Example 19. The computer-implemented method of any of Examples 1 through 18, wherein the particular zone configuration comprises boundaries of the zones.

Example 20. The computer-implemented method of any of Examples 1 through 19, wherein the particular zone configuration comprises one or more zip codes associated with each zone.

Example 21. The computer-implemented method of any of Examples 1 through 20, wherein the particular zone configuration comprises the demand nodes assigned to each zone.

Example 22. The computer-implemented method of any of Examples 1 through 21, wherein the particular zone configuration comprises the supply nodes assigned to each zone.

Example 23. A system comprising memory and one or more processors communicatively coupled to the memory, the one or more processors configured to perform the method of any one of Examples 1 through 22.

Example 24. One or more non-transitory computer-readable storage media including instructions that, when executed by one or more processors, cause the one or more processors to perform the method of any of Examples 1 through 22.

### Additional Considerations

The following considerations also apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

It should also be understood that, unless a term is expressly defined in this patent using the sentence “As used herein, the term ‘XYZ’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, which is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112 (f).

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of “a” or “an” is employed to describe elements and components of the embodiments herein. This is done merely for convenience and to give a general sense of the disclosure. This description should be read to include one or at least one and the singular also includes the plural unless it is obvious that it is meant otherwise.

Upon reading this disclosure, those of skill in the art will appreciate still additional alternative structural and functional designs for implementing the concepts disclosed herein, through the principles disclosed herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed:

1. A computer-implemented method comprising:
generating, by one or more processors, a plurality of zone configurations, at least by applying a machine learning (ML) model to demand node data indicating locations of a plurality of demand nodes, wherein each zone configuration of the plurality of zone configurations includes a respective set of zones among a plurality of sets of zones;
determining, by the one or more processors, a plurality of respective costs for the plurality of zone configurations, wherein determining the plurality of respective costs for each zone configuration of the plurality of zone configurations comprises:
determining supply node allocations for the respective set of zones included in the zone configuration based on times or distances between (i) demand nodes, of the plurality of demand nodes, that are associated with the respective set of zones and (ii) supply nodes, of a plurality of supply nodes, that are eligible for the respective set of zones, and
determining a respective cost, of the plurality of respective costs, for the zone configuration based on (i) the times or distances and (ii) the supply node allocations;
selecting, by the one or more processors, a particular zone configuration, from among the plurality of zone configurations, based on the plurality of respective costs; and
storing in a memory, by the one or more processors, one or more data objects representing the particular zone configuration.

2. The computer-implemented method of claim 1, wherein determining the supply node allocations for the respective set of zones included in the zone configuration based on the times or distances comprises:
summing the times or distances from (i) the demand nodes to a zone centroid or a zone medoid for the respective set of zones and (ii) the supply nodes to the zone centroid or the zone medoid for the respective set of zones.

3. The computer-implemented method of claim 1, wherein the ML model comprises a k-means clustering model.

4. The computer-implemented method of claim 1, wherein each zone configuration of the plurality of zone configurations consists of a different number of zones.

5. The computer-implemented method of claim 1, wherein determining the supply node allocations for the respective set of zones included in the zone configuration comprises:
verifying that an allocation of supply nodes to the respective set of zones does not exceed a maximum count for each supply node, wherein the maximum count is a maximum number of demand nodes that can be assigned to a single supply node.

6. The computer-implemented method of claim 1, wherein determining the supply node allocations for the respective set of zones included in the zone configuration comprises:
verifying that an allocation of supply nodes to the respective set of zones equals or exceeds a minimum count for each supply node, wherein the minimum count is a minimum number of demand nodes that can be assigned to a single supply node.

7. The computer-implemented method of claim 1, further comprising:
determining, by the one or more processors and for one or more of the supply nodes, a zone assignment based on the particular zone configuration, wherein the zone assignment comprises a single zone of the plurality of respective zones assigned to each of the one or more supply nodes; and
causing, by the one or more processors, a display to present a visual indication of the zone assignment.

8. The computer-implemented method of claim 1, wherein determining the supply node allocations for the respective set of zones included in the zone configuration comprises minimizing a total cost for the zone configuration using an objective function.

9. A system comprising:
a memory; and
one or more processors communicatively coupled to the memory, the one or more processors configured to:
generate a plurality of zone configurations at least by applying a machine learning (ML) model to demand node data indicating locations of a plurality of demand nodes, wherein each zone configuration of the plurality of zone configurations includes a respective set of zones among a plurality of sets of zones;
determine a plurality of respective costs for the plurality of zone configurations, wherein determining the plurality of respective costs for each zone configuration of the plurality of zone configurations comprises:
determining supply node allocations for the respective set of zones included in the zone configuration based on times or distances between (i) demand nodes, of the plurality of demand nodes, that are associated with the respective set of zones and (ii) supply nodes, of a plurality of supply nodes, that are eligible for the respective set of zones, and
determining a respective cost, of the plurality of respective costs, for the zone configuration based on (i) the times or distances and (ii) the supply node allocations;
select a particular zone configuration, from among the plurality of zone configurations, based on the plurality of respective costs; and
store in the memory one or more data objects representing the particular zone configuration.

10. The system of claim 9, wherein determining the supply node allocations for the respective set of zones included in the zone configuration based on the times or distances comprises:
summing the times or distances from (i) the demand nodes to a zone centroid or a zone medoid for the respective set of zones and (ii) the supply nodes to the zone centroid or the zone medoid for the respective set of zones.

11. The system of claim 9, wherein the ML model comprises a k-means clustering model.

12. The system of claim 9, wherein each zone configuration of the plurality of zone configurations consists of a different number of zones.

13. The system of claim 9, wherein determining the supply node allocations for the respective set of zones included in the zone configuration comprises:
verifying that an allocation of supply nodes to the respective set of zones does not exceed a maximum count for each supply node, wherein the maximum count is a maximum number of demand nodes that can be assigned to a single supply node.

14. The system of claim 9, wherein determining the supply node allocations for the respective set of zones included in the zone configuration comprises:
verifying that an allocation of supply nodes to the respective set of zones equals or exceeds a minimum count for each supply node, wherein the minimum count is a minimum number of demand nodes that can be assigned to a single supply node.

15. The system of claim 9, wherein the one or more processors are further configured to:
determine, for one or more of the supply nodes, a zone assignment based on the particular zone configuration, wherein the zone assignment comprises a single zone of the plurality of respective zones assigned to each of the one or more supply nodes; and
cause a display to present a visual indication of the zone assignment.

16. The system of claim 9, wherein determining the supply node allocations for the respective set of zones included in the zone configuration comprises minimizing a total cost for the zone configuration using an objective function.

17. One or more non-transitory computer-readable storage media including instructions that, when executed by one or more processors, cause the one or more processors to:
generate a plurality of zone configurations at least by applying a machine learning (ML) model to demand node data indicating locations of a plurality of demand nodes, wherein each zone configuration of the plurality of zone configurations includes a respective set of zones among a plurality of sets of zones;
determine a plurality of respective costs for the plurality of zone configurations, wherein determining the plurality of respective costs for each zone configuration of the plurality of zone configurations comprises:
determining supply node allocations for the respective set of zones included in the zone configuration based on times or distances between (i) demand nodes, of the plurality of demand nodes, that are associated with the respective set of zones and (ii) supply nodes, of a plurality of supply nodes, that are eligible for the respective set of zones, and
determining a respective cost, of the plurality of respective costs, for the zone configuration based on (i) the times or distances and (ii) the supply node allocations;
select a particular zone configuration, from among the plurality of zone configurations, based on the plurality of respective costs; and
store in a memory one or more data objects representing the particular zone configuration.

18. The one or more non-transitory computer-readable storage media of claim 17, wherein determining the supply node allocations for the respective set of zones included in the zone configuration based on the times or distances comprises:
summing the times or distances from (i) the demand nodes to a zone centroid or a zone medoid for the respective set of zones and (ii) the supply nodes to the zone centroid or the zone medoid for the respective set of zones.

19. The one or more non-transitory computer-readable storage media of claim 17, wherein the ML model comprises a k-means clustering model.

20. The one or more non-transitory computer-readable storage media of claim 17, wherein each zone configuration of the plurality of zone configurations consists of a different number of zones.

21. The one or more non-transitory computer-readable storage media of claim 17, wherein determining the supply node allocations for the respective set of zones included in the zone configuration comprises:
verifying that an allocation of supply nodes to the respective set of zones does not exceed a maximum count for each supply node, wherein the maximum count is a maximum number of demand nodes that can be assigned to a single supply node.

22. The one or more non-transitory computer-readable storage media of claim 17, wherein determining the supply node allocations for the respective set of zones included in the zone configuration comprises:
verifying that an allocation of supply nodes to the respective set of zones equals or exceeds a minimum count for each supply node, wherein the minimum count is a minimum number of demand nodes that can be assigned to a single supply node.

23. The one or more non-transitory computer-readable storage media of claim 17, wherein the instructions further cause the one or more processors to:
determine, for one or more of the supply nodes, a zone assignment based on the particular zone configuration, wherein the zone assignment comprises a single zone of the plurality of respective zones assigned to each of the one or more supply nodes; and
cause a display to present a visual indication of the zone assignment.

24. The one or more non-transitory computer-readable storage media of claim 17, wherein determining the supply node allocations for the respective set of zones included in the zone configuration comprises minimizing a total cost for the zone configuration using an objective function.
